(19) United States
(12) Patent Application Publication
(10) Pub. No.: US 2011/0046448 A1
(43) Pub. Date: Feb. 24, 2011

(54) Cardiac tissue retractor with associated valve cusp depressor

(76) Inventors: Anthony Paolitto, Montreal (CA); Valerio Valentini, Montreal (CA)

Correspondence Address: Anthony PAOLITTO;CORONEO INC., Suite 514, 9250 avenue du Parc, Montreal, QC, H2N 1Z2, CA

(21) Appl. No.: 12/805,538

(22) Filed: Aug. 3, 2010

Related U.S. Application Data
(60) Provisional application No. 61/213,960, filed on Aug. 3, 2009.

(51) Int. Cl. A61B 1/32; A61B 19/00

(52) U.S. Cl. 600/201; 128/898

(57) ABSTRACT

A tissue retracting apparatus for use in cardiac surgery having a first tissue retracting member configured and sized to retract a cardiac tissue of the patient's heart in a manner to obtain surgical access to a target heart valve located within an internal heart cavity, and a second tissue retracting member configured and sized to retract, depress or displace a valve cusp tissue of the target heart valve in a manner to obtain surgical access beyond the target heart valve. In use, the second tissue retracting member is operatively couplable to the first retracting member, so that the first and second retracting members cooperate together to collectively allow the simultaneous retraction of both (i) a cardiac tissue of the patient's heart, and (ii) a valve cusp of the target valve while a surgical intervention can be carried out on either the target heart valve or a subvalvular structure of said target heart valve.

This application claims the benefits of United States Provisional Patent Application 61/213,960 filed Aug. 3, 2009.

### FIELD OF THE INVENTION

The present invention relates to the field of cardiac surgical instruments and more specifically, to cardiac tissue retractors that are adapted for use in valve surgery to retract a portion of a patient's heart in order to access a target heart valve found therein, said tissue retractor also provided with a means or member that is configured to depress, displace or retract a valve cusp of said target valve during said use.

### BACKGROUND OF THE INVENTION

Current tissue retractors, especially in cardiac surgery, are typically of a fixed geometry. They are most commonly configured at the tissue-retracting end with either a “basket” type configuration made from spaced apart wire frame members, or with an uninterrupted and shaped tissue contacting surface or blade that engages the cardiac or heart tissue to be retracted. These retractors are most typically employed to retract the cardiac tissue comprising the left atrium of the heart during a surgery on the mitral heart valve, or the cardiac tissue comprising the right atrium during a surgery on the tricuspid heart valve. During retraction of said atria by said known retractors, the latter are not configured with an additional means or separate member specially configured to independently retract a cusp portion of the target valve (or are not configured with a distinct portion to specifically retract a cusp portion of the target valve), in order to advantageously provide improved surgical and visual access across the target valve to either the subvalvular structures thereof or the supravalvular space thereabove, depending on whether the tissue retractor is being deployed to retract cardiac tissue from upstream or downstream of said target valve.

In cardiac surgery requiring the retraction of heart tissue, for instance in a mitral valve surgery practiced via a left atrial approach, commonly used retractor platforms include the “Cosgrove-type” and “Carpentier-type” retractor platforms. In using the “Cosgrove-type” retractor platform (see FIG. 1), generally three fixed geometry basket-type tissue retractors are deployed to retract the incised cardiac tissue of the left atrium to gain proper access to the target heart valve (i.e. the mitral valve) requiring the surgical procedure or intervention. Each of these tissue retractors is independently mounted or secured to a sternal retractor, or stable surgical platform, to achieve the desired retraction of the atrial incision and to obtain surgical access to the target mitral valve. In using the “Carpentier-type” retractor platform (see FIG. 2), generally two tissue retractors, each having a fixed-shape tissue contacting surface, are deployed to retract the left atrium and mounted to a sternal retractor. Neither the Cosgrove nor the Carpentier tissue retractors are provided with a separate cooperating member or are provided with a distinct portion intently configured and sized to retract a valve cusp of the target valve. Neither the Cosgrove nor Carpentier known tissue retractors are provided with an additional means or member that is operatively couplable to the tissue retractor to retract, displace, or depress a valve cusp when the atrial tissue retractor is engaged with atrial tissue and retracting same.

Recently, with the advent of minimally invasive cardiac surgery gaining in popularity, the size of the retracted thoracic opening or surgical window, and the size of the surgical access incision into the patient's heart are being progressively reduced. Having an independently mounted atrial retractor, and requiring a surgical assistant having to depress a target valve cusp with a makeshift cusp depressor through a limited access port in patient's thorax, without encumbering the surgeon's vision or access, makes such minimally invasive procedures impractical or in some cases impossible to achieve, given the relatively smaller size of the surgical window.

Currently known cardiac tissue retractors, whether deployed through a sternotomy access or intercostal approach for surgery on the mitral valve via a left atrial approach, are not advantageously provided with a complementary means or additional member or a provision having a specifically designated configuration to also retract a cusp of the target valve. Such a cooperating additional means or member or provision would allow the surgeon to displace, for instance, the anterior cusp of the mitral valve so as to ergonomically gain access to the subvalvular apparatus of the mitral valve located within the left ventricle. This access is advantageous in allowing the surgeon to repair chordae, add artificial chordae between papillary muscle and leaflet or cusp, or effect a surgical intervention on the papillary muscles or some other part of the target valve subvalvular structure or a part of the ventricle, while the cardiac tissue defining the left atrium is also being simultaneously retracted by the cardiac tissue retractor.

Currently, when the above described interventions on the subvalvular structures of the mitral valve are required, the surgeon must retract or displace the cusp of the target valve with a surgical instrument such as a forceps or other surgical instrument not specifically designed for cusp displacement. The surgeon must then carry out a precise gesture on the subvalvular structures of the target valve, while keeping the cusp or a portion thereof displaced, depressed or retracted in order to have proper vision on the subvalvular structures. A separate independent make-shift “depressor” may also be deployed, but it must be held by a surgical assistant or alternatively wedged in an ad hoc manner between other instruments within the surgical field (i.e. chest retractor) or attached to a surgical drape or even to parts of patient's anatomy.

### SUMMARY OF THE INVENTION

Thus, it is a first object of the present invention to provide a tissue retracting apparatus having a first tissue retracting member configured and sized to retract a cardiac tissue of the patient's heart (for example, a cardiac tissue defining one of the walls of the heart chamber) to obtain surgical access to a target heart valve, and a second tissue retracting member configured and sized to retract, depress or displace a valve cusp tissue of said target heart valve, said second tissue retracting member being operatively couplable to said first retracting member, in use, so that said first and second retracting members cooperate to collectively allow the simultaneous retraction of both (i) a cardiac tissue of the patient's heart, and (ii) a valve cusp of said target valve while a surgical intervention takes place on said target heart valve or a subvalvular structure of said target heart valve.

Thus, it is a second object of the present invention to provide a tissue retractor configured to retract a portion of the patient's heart (i.e. non-target tissue), said retractor being provided with a cooperating cusp depressor means or member that is operatively couplable to said cardiac tissue retractor during use, said coupling allowing an additional or simultaneous retraction, displacement, or depression of a cusp of a target valve (ie target tissue) while said non-target heart tissue is also being retracted.

It is a further object of the present invention to provide a tissue retracting apparatus comprising a cardiac tissue retractor, said cardiac tissue retractor having a plurality of adaptable tissue-retracting or tissue-engaging blades, said blades configured and sized to retract a cardiac tissue of the patient's heart to obtain surgical access to a target cardiac valve, said plurality of tissue-retracting blades being adjustable or movable relative to each other between a blade closed configuration whereby said blades are in proximity to each other and a blade open configuration whereby said blades are in a spaced apart spatial relationship so that, in use, the cardiac tissue retractor may be customized or tailored to suit the specific anatomy of the patient or the specific geometry of a surgical incision with a desired blade spatial relationship, said tissue retracting apparatus further comprising a cooperating cusp depressor means or member that is operatively couplable to at least one of said tissue-engaging blades during use, said cusp depressor configured and sized to retract, displace or depress a cusp of said target valve when it is coupled to said at least one of said tissue-engaging blades whereby with the deployment of said tissue retracting apparatus, said cardiac tissue retractor and said cusp depressor cooperate to provide a retraction of said cardiac tissue and a complementary simultaneous depression of said target valve cusp.

It is a further object of the present invention to be able to mount a cardiac tissue retractor to a stable surgical platform in order to retract a cardiac tissue so as to obtain proper surgical access to a target cardiac valve being operated on, and in situ, during use, operatively couple a valve cusp depressor to said cardiac tissue retractor, and secure the position of said cusp depressor to said cardiac tissue retractor, thus avoiding the need to independently and separately mount said cusp depressor to said surgical platform through a separate surgical set-up.

These and other objects of the present invention will become apparent from the description of the present invention and its preferred embodiments which follows.

### BRIEF DESCRIPTION OF THE DRAWINGS

For better understanding of the present invention and to show more clearly how it may be carried into effect, reference will now be made by way of illustration and not of limitation to the accompanying drawings, which show a tissue retractor apparatus according to preferred embodiments of the present invention, and in which:

FIG. 1 is a perspective view of a prior art surgical platform commonly known as a “Cosgrove-type” retractor platform for a sternotomy approach to the mitral valve MV;

FIG. 2 is a perspective view of a prior art surgical platform commonly known as a “Carpentier-type” retractor platform for a sternotomy approach to the mitral valve MV;

FIG. 3 is a perspective view of a prior art surgical retractor commonly known as a “Heartport-type” blade retractor for an intercostal approach to the mitral valve MV;

FIG. 4 is a perspective view of a tissue retracting apparatus 1 mounted to a chest retractor 99 and comprising a cardiac tissue retractor 40 having a plurality of movable tissue-retracting blades 41, 42, 43 retracting a left atrium cardiac tissue and a cusp depressor 50 prior to being coupled to one of said tissue-retracting blades 42 to depress a mitral valve cusp, according to a preferred embodiment of the present invention;

FIG. 5A is a perspective view of the tissue retracting apparatus 1 illustrated in FIG. 4, with the cardiac tissue retractor 40 decoupled from its housing 20 and with the tissue-retracting blades 41, 42, 43 in a closed-blade configuration 44 to facilitate the insertion of said blades through an intercostal access port IAP into the patient's thoracic cavity;

FIG. 5B is a close up view of a the tissue retracting apparatus 1 of FIG. 4 illustrating the cardiac tissue retractor 40 with movable plurality of tissue-retracting blades 41, 42, 43 in an open-blade configuration 45 for retracting a cardiac tissue, and a valve cusp depressor 50 prior to the latter being coupled to the middle blade 42 of said cardiac tissue retractor 40 in order to depress a target valve cusp;

FIG. 5C is a close up view of a the tissue retracting apparatus 1 of FIG. 4 illustrating the valve cusp depressor 50 being coupled to the middle blade 42 of the cardiac tissue retractor 40 at a depressor-to-retractor interface 60 and prior to the distal end 52 of said cusp depressor engaging a target valve cusp;

FIG. 5D is a close up view of a the tissue retracting apparatus 1 of FIG. 4 illustrating the cusp depressor being fully engaged with the middle blade 42 of the cardiac tissue retractor 40 with the distal end 52 of the cusp depressor 50 extending generally beyond the distal most end 421 of blade 42 in order to engage a target valve cusp, said cusp depressor 50 being securely locked in place by a locking means 70 located between the cardiac tissue retractor 40 and cusp depressor 50 at the proximal end 53 thereof;

FIG. 6 is a close-up perspective view of the tissue retracting apparatus 1 of FIG. 4 illustrating the three blades 41, 42, 43 of the cardiac tissue retractor 40 engaged with and retracting a left atrium wall LAWT of the patient's heart HRT, and the cusp depressor 50 coupled to the cardiac tissue retractor middle blade 42 and simultaneously depressing the anterior cusp AC of the target mitral valve MV providing an access to the chordae and subvalvular apparatus of the mitral valve MV located within the left ventricle of the patient's heart HRT;

FIG. 7 is a perspective view of the tissue retracting apparatus 1 according to a preferred embodiment of the present invention; the cusp depressor 50 is illustrated demounted from one of the blades 41, 42 of the cardiac tissue retractor 40;

FIG. 8 is a partially exploded view of the tissue retracting apparatus 1 of FIG. 7 illustrating the actuating member in the nature of a flexible cable 11 unassembled from the housing 20 of the tissue retracting apparatus 1;

FIG. 9 is a close up bottom view of the tissue retracting apparatus 1 of FIG. 7 illustrating the cardiac tissue retractor 40 and linkage mechanism 30 engaged with the distal ball end 110 of the actuating cable 11 and prior to the linkage mechanism 30 being engaged with the tubular housing 20 at the housing coupling interface 22;

FIG. 10 is a cross-sectional view through a portion of the tissue retracting apparatus 1 of FIG. 7 illustrating a cut away view through the housing proximal end 23 with the actuator 10 engaging a housing threaded portion 25 thereof with said linkage mechanism 30 engaged at the housing coupling interface 22, said actuator 10 movable relative to said housing threaded portion 25 between a first threaded position 153 and a second threaded portion 154 by the rotation of the actuator 10, resulting in the movement of said tissue-retracting blades 41, 42, 43 between a closed blade 44 and open blade configuration 45, respectively.

FIG. 11A is an assembly view illustrating the freedom of movement 54, 55 of the cusp depressor 50 relative to the cardiac tissue retractor 40 when said depressor is coupled to the cardiac tissue retractor 40 at the depressor-to-retractor interface 60 and prior to the depressor 50 being fully seated and locked relative to the cardiac tissue retractor 40.

FIG. 11B is an assembly view illustrating the cusp depressor 50 in its fully seated position relative to the cardiac tissue retractor 40, the distal end 52 of said depressor extending a predetermined desirable distance L1 beyond the distal most end 421 of cardiac tissue retractor 40, and a distance D1 below the distal most end 421 of cardiac tissue retractor;

FIG. 11C is a top view of FIG. 11B illustrating a variant of cusp depressor 50 configured with a predetermined and desirable offset distance W relative to the longitudinal axis 29 of tissue retracting apparatus 1;

FIG. 12A illustrates a variant of the cusp depressor 50 illustrated in FIG. 11B, the latter configured with a bulbous terminal end 57 and a smaller distance D2 below the distal most end 421 of cardiac tissue retractor.

FIGS. 12B-12H illustrate variant configurations of cusp depressor terminal ends 52 including a hooked terminal end 521 (FIG. 12B), a slotted terminal end 522 (FIG. 12C), an open body cusp depressor 523 (FIG. 12D), a textured terminal end 524 (FIG. 12E), a clear plastic cusp depressor 525 (FIG. 12F), a cusp depressor configured to house an illuminating or vision-system fiber optic bundle 526 (FIG. 12G), and a cusp depressor configured with a fluid transfer channel therethrough in the nature of a carbon dioxide gas CO2 transfer passageway 527 (FIG. 12H);

FIG. 13A-13B, according to a second embodiment of the present invention, illustrate a tissue retracting apparatus 2 comprising a single, relatively wider, fixed-geometry cardiac tissue retractor 400 and a couplable cusp depressor 50.

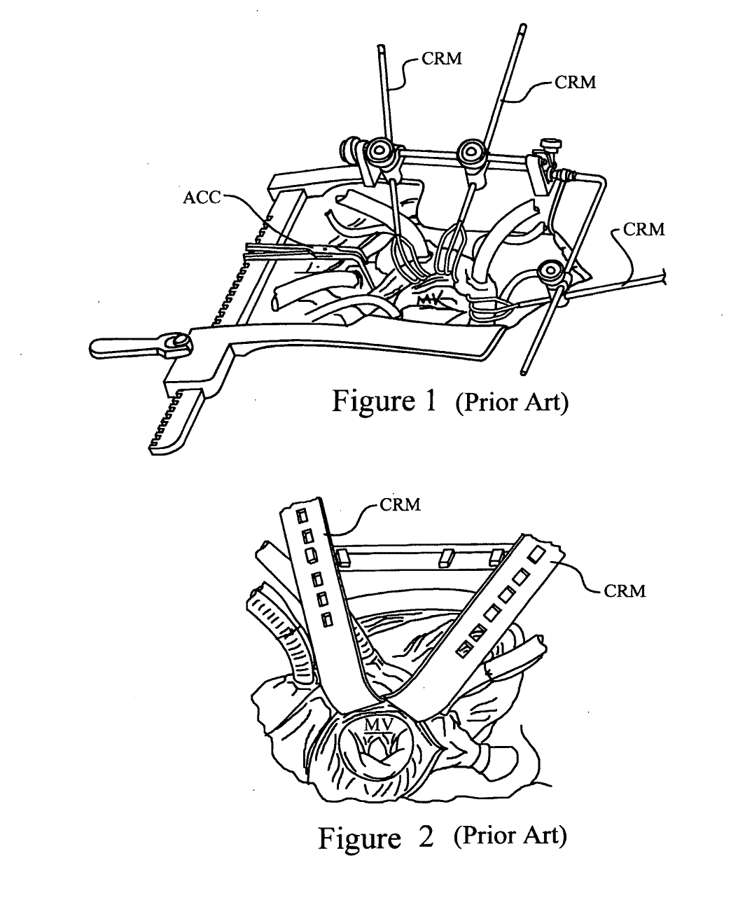

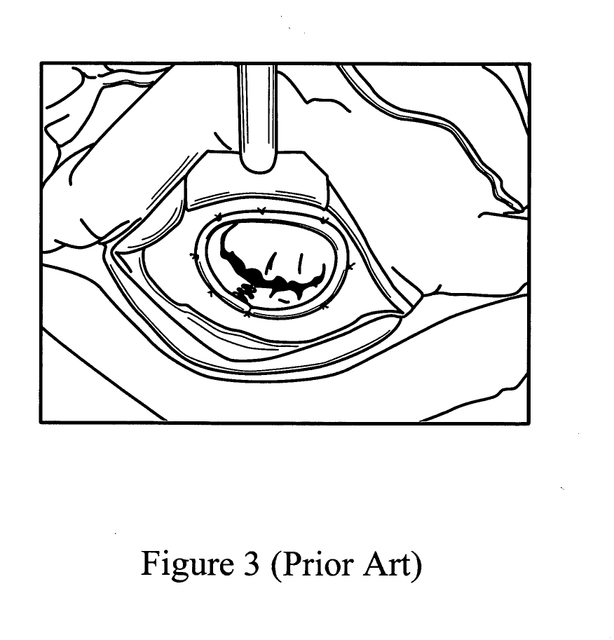

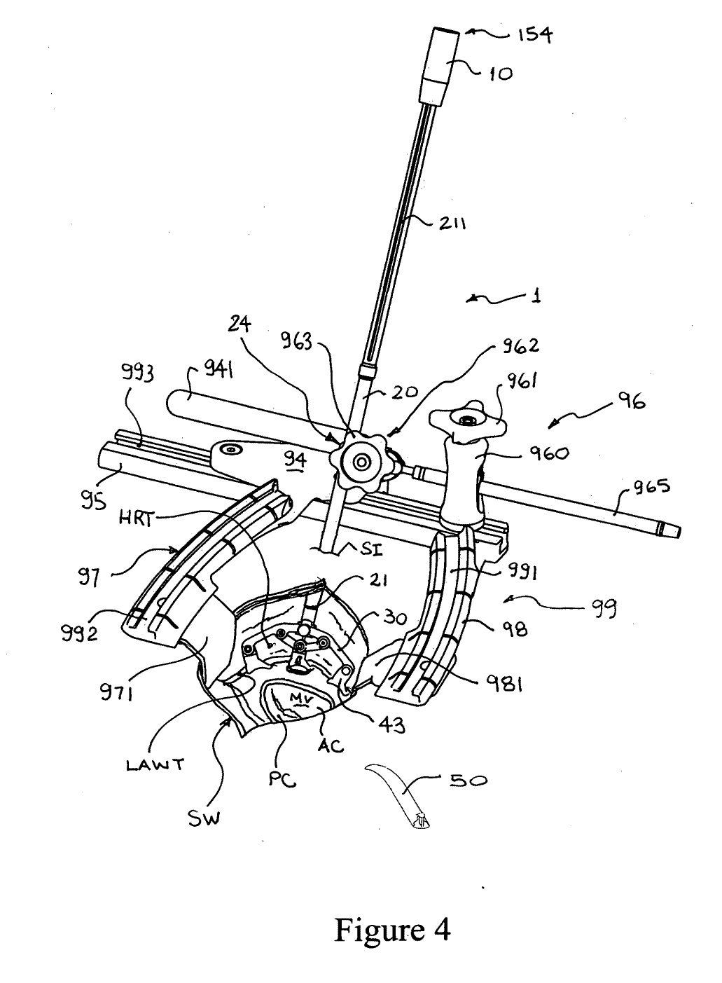

### DETAILED DESCRIPTION OF THE INVENTION

The invention will be described in the context of a cardiac valve surgery performed on the mitral valve of the patient. It is understood that the concepts and principles of the invention may be applied to tissue retracting apparatus used to perform cardiac surgery on the other cardiac valves (i.e. pulmonary, tricuspid, and aortic) without departing from the spirit of the invention.

The heart is contained within a patient's thorax or thoracic cavity, and is located beyond a structural ribcage. The heart includes a number of internal cavities through which blood flows and which are associated with a heart valve. Included in these internal cavities are the heart chambers (left atrium, right atrium, left ventricle, right ventricle). Each of the heart chambers is delimited by a number of chamber-defining walls and inner chamber partitions or septal walls. As well, each of the heart chambers is delimited by at least one cardiac valve to control passage of blood flow through the chamber in a synchronized manner with each heart beat. Apart from the heart chambers and included in these internal cavities are the passageways or regions within the cardiac anatomy which are immediately adjacent or associated with a heart valve. For instance, the aortic root located just downstream and above the aortic valve is one such cavity which surgeons routinely access when performing a surgical procedure on the aortic valve (or the ascending aorta and the sinuses of Valsalva). The different heart valves (aortic, mitral, tricuspid, or pulmonary) have at least one valve cusp that is displaced between a valve closed and valve open configuration to selectively restrict or allow passage of blood therethrough.

The patient's heart is comprised of different cardiac tissues including tissue of the aorta, tissue of the vena cavae, tissue of the pulmonary veins and arteries, tissue of the left and right atria, tissue of the left and right ventricles, tissue of the atrial septum, and tissue of the ventricular septum. For the purposes of this description of the invention, the term “cardiac tissue” will include all tissues of the heart that may need to be retracted in order to gain surgical or visual access to a heart valve; that is, the “target cardiac valve”. The terms “valve cusp” or “cusp tissue” of “valve tissue” will refer specifically to the tissue defining the valve cusps of the target cardiac valve.

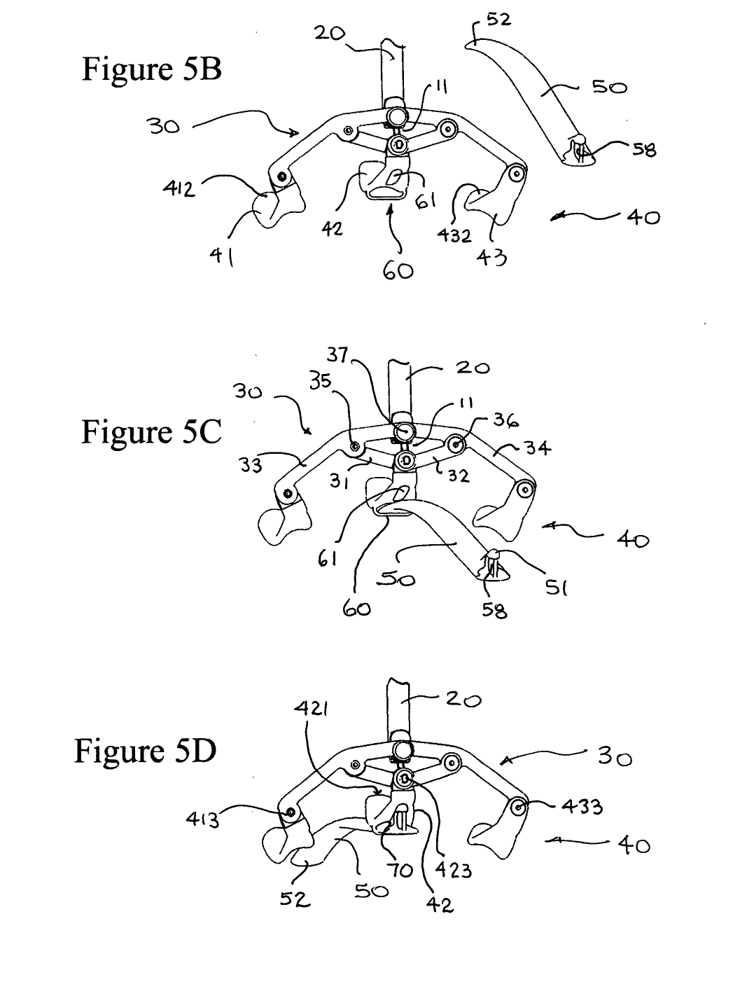

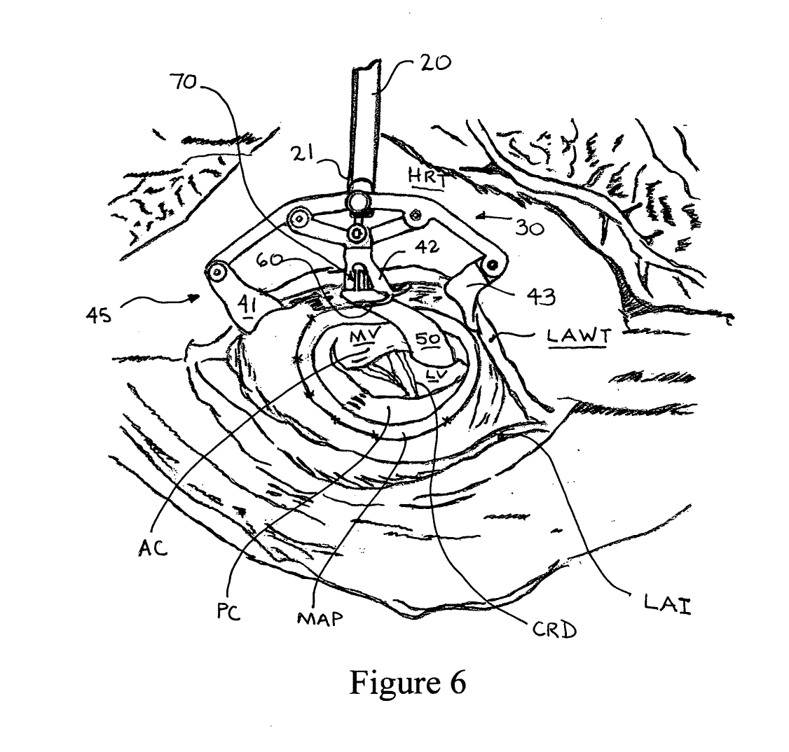

Referring to FIGS. 5A and 6, a patient's heart HRT is accessed via an intercostal access port IAP in a thoracic cavity TC. A left atriotomy incision or left atrial incision LAI in the left atrium of the heart HRT provides surgical and visual access to said mitral valve MV, and more specifically to the anterior AC and posterior PC valve leaflets or cusps. Beyond the cusps is the left ventricle LV which houses the subvalvular apparatus of said mitral valve MV including a plurality of chordae tendinae CRD attached at one end to the underside of the mitral valve cusps AC, PC and at a second end, also attached to the papillary muscles (not shown) located deeper within the left ventricle LV.

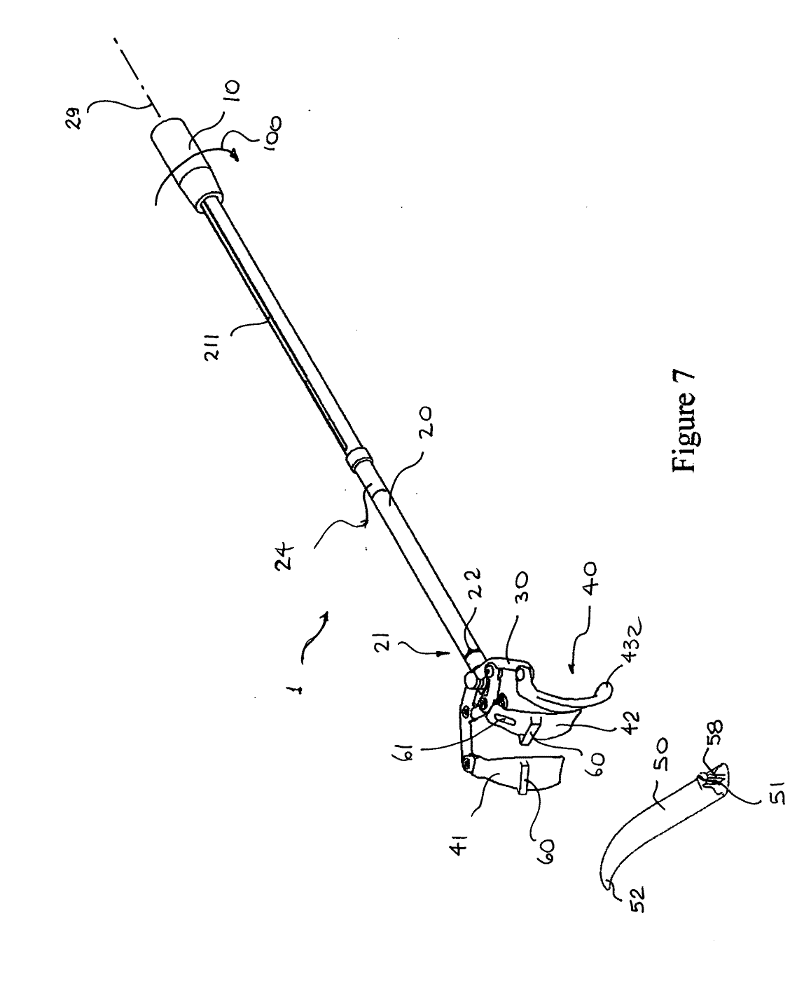

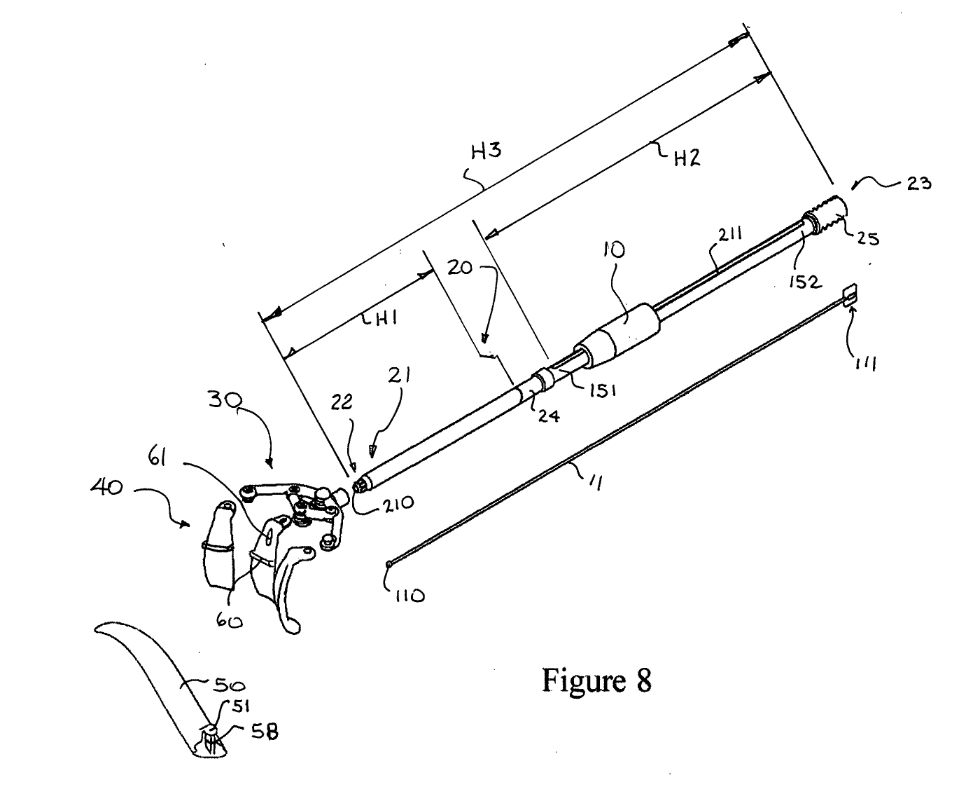

Referring to FIGS. 7 and 8, a preferred embodiment of a tissue retracting mechanism, assembly or apparatus 1 is comprised of a first retracting member or cardiac tissue retractor 40, a second retracting member, cusp retractor or cusp depressor 50, a linkage assembly or mechanism 30, a retractor housing 20, and an actuator 10.

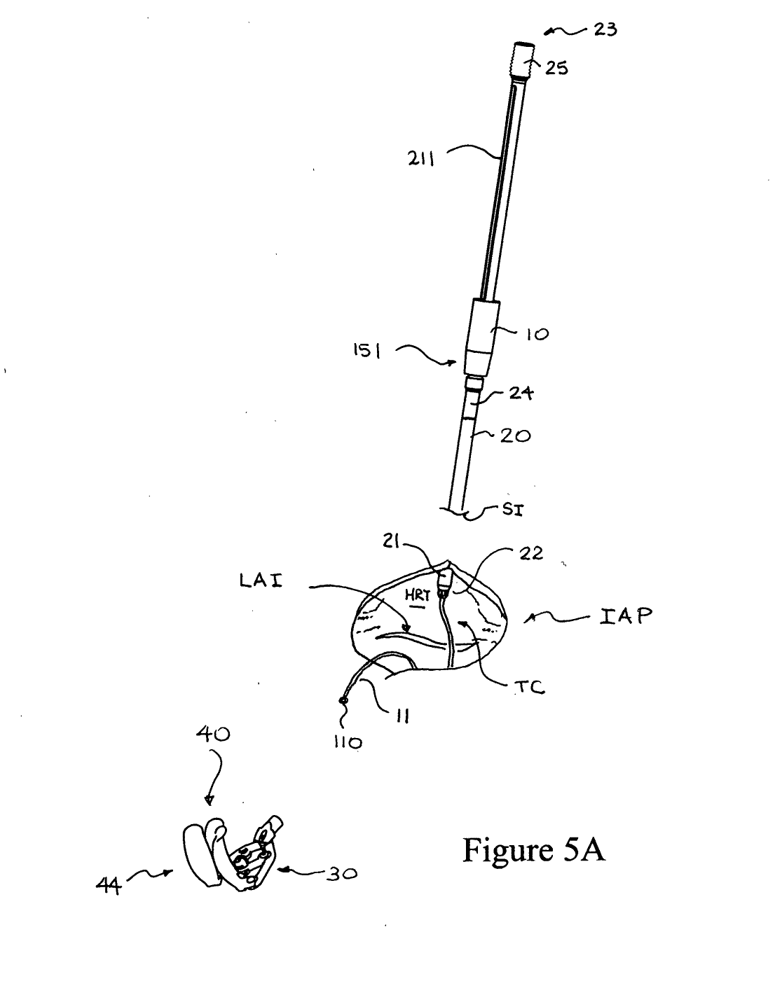

As illustrated in FIG. 4, tissue retracting mechanism of apparatus 1 is preferably mounted to a substantially stable surgical platform, such as a chest retractor, or more specifically, an intercostal thoracic retractor 99 via an instrument positioning arm 96. Thoracic retractor 99 is comprised of a first, movable spreader arm 97 and a second, fixed spreader arm 98. Arms 97 and 98 are provided with blades 971, 988 respectively, said blades being configured and sized to spread apart two adjacent ribs of the patient's ribcage, in order to obtain surgical access to the underlying thoracic cavity TC and the patient's heart HRT located therewithin. Arm 97 moves relative to arm 98 along rack bar 95 when crank mechanism 94 is actuated by a rotation of pinion 941, and as such the relative lateral spacing between blades 971, 981, and the resulting surgical window SW may be controlled.

Instrument positioning arm 96 includes a first mechanical joint or clamp 960 which is provided with a key member or fitting (not shown) designed to slidingly engage or mate with perimeter rails 991, 992 or 993 of thoracic retractor 99. As such, joint 960 (and consequently arm 96) may be variably mounted anywhere along perimeter rails 991, 992 or 993. As well, mechanical joint 960 secures the position and orientation of arm member or rod 965 relative to thoracic retractor 99, and the position of mechanical joint 960 along anyone of said perimeter rails, when knob 961 is tightened. Instrument positioning arm 96 also includes a second mechanical joint or clamp 962 which is configured to engage with and clamp tissue retracting apparatus 1. Tissue retracting apparatus 1 is provided with an apparatus-mounting-interface, or mounting seat 24 which advantageously allows said apparatus 1 to be engaged within said clamp member 962. Clamp member 962 provides multiple motion degrees of freedom thus allowing the surgeon to vary the angular orientation between housing 20 and rod 965. Tightening clamp knob 963 results in securing said angular orientation. As such, through instrument positioning arm 96, the position and orientation of tissue retracting apparatus 1 may be secured in desired spatial relationship relative to thoracic retractor 99 (and also the patient's thorax which retractor 99 is engaged with) when clamp knobs 961, 963 are tightened. This allows the surgeon to impart the desired tissue retraction to a cardiac tissue and then secure this retraction load by clamping the tissue retracting apparatus 1 to thoracic retractor 99 in the optimum retracting position and orientation.

It is understood that tissue retracting apparatus 1 may alternatively be mounted to other types of surgical platforms via positioning arm 96 or even other types of instrument positioning arms. For instance, tissue retracting apparatus 1 may be mounted to a surgical table via a multi-jointed articulating surgical arm well known in the field of endoscopic surgery. For instance, tissue retracting apparatus 1 may be mounted to a sternotomy chest retractor configured with a perimeter rail 991, 992, or 993 via instrument positioning arm 96.

Referring to FIGS. 4-9, cardiac tissue retractor 40 is preferably comprised of a plurality of cardiac tissue-engaging or cardiac tissue-retracting fingers or blades 41, 42, 43. As illustrated in FIGS. 4 and 6, said tissue-retracting blades are suitably configured and appropriately sized to engage with and retract a cardiac tissue, in this case, portion of the incised left atrium or left atrial wall tissue LWAT, thereby providing the surgeon with surgical access to the mitral valve MV (i.e. the target heart valve) via a left atrial incision LAI. Accordingly, terminal blade ends 412, 422, 432 are bent and configured with a hook-like geometry adapted to hook the LAWT and minimize slipping of said cardiac tissue relative to said blades 41, 42, 43 when a retracting load is applied to tissue retracting apparatus 1. As well, said terminal ends are also profiled to be blunt and atraumatic so as to not pierce through the cardiac tissue being retracted. Preferably, blades 41, 42, 43 are sized with a blade length BL from 1.2 to 2.4 inches (30 to 60 mm), and a blade width BW from 0.275 to 0.470 inches (7 to 12 mm). Other sizes are also suitable, depending on the size of the patient's heart HRT and size of left atrium to be retracted.

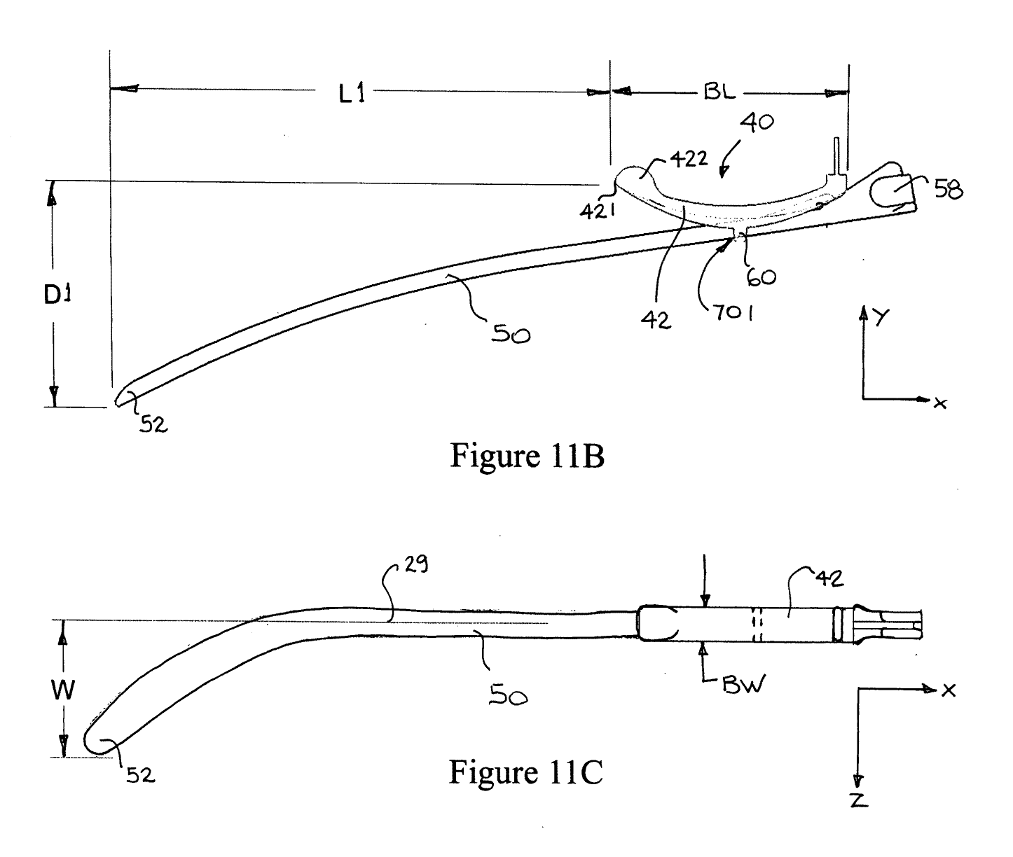

Referring to FIGS. 11A-11B, second retracting member in the nature of a cusp retractor, cusp depressing member or cusp depressor 50 is generally elongate extending between a first proximal depressor end 53 and a second distal depressor end 52. Cusp depressor 52 is configured and sized to displace, depress, or retract a cusp of a target heart valve (for example, the mitral valve MV as illustrated in FIG. 6), or a portion of a target heart valve cusp, when said cusp depressor 50 is coupled to said cardiac tissue retractor 40, according to a surgical method that will be described below. Cusp depressor 50 is preferably generally arcuate in shape, ideally suited to extend between said proximal 53 and distal 52 ends in a configuration that is least obstructive to the surgeon's view and less encumbering to the surgical access to a target heart valve when said depressor 50 is connected to said cardiac tissue retractor 40, and said distal end 52 extends beyond the target heart valve. For instance, as illustrated in FIG. 11B, distal end 52 extends a predetermined desirable distance L1 beyond, a depth or distance D1 below, the distal most end 421 of blade 42.

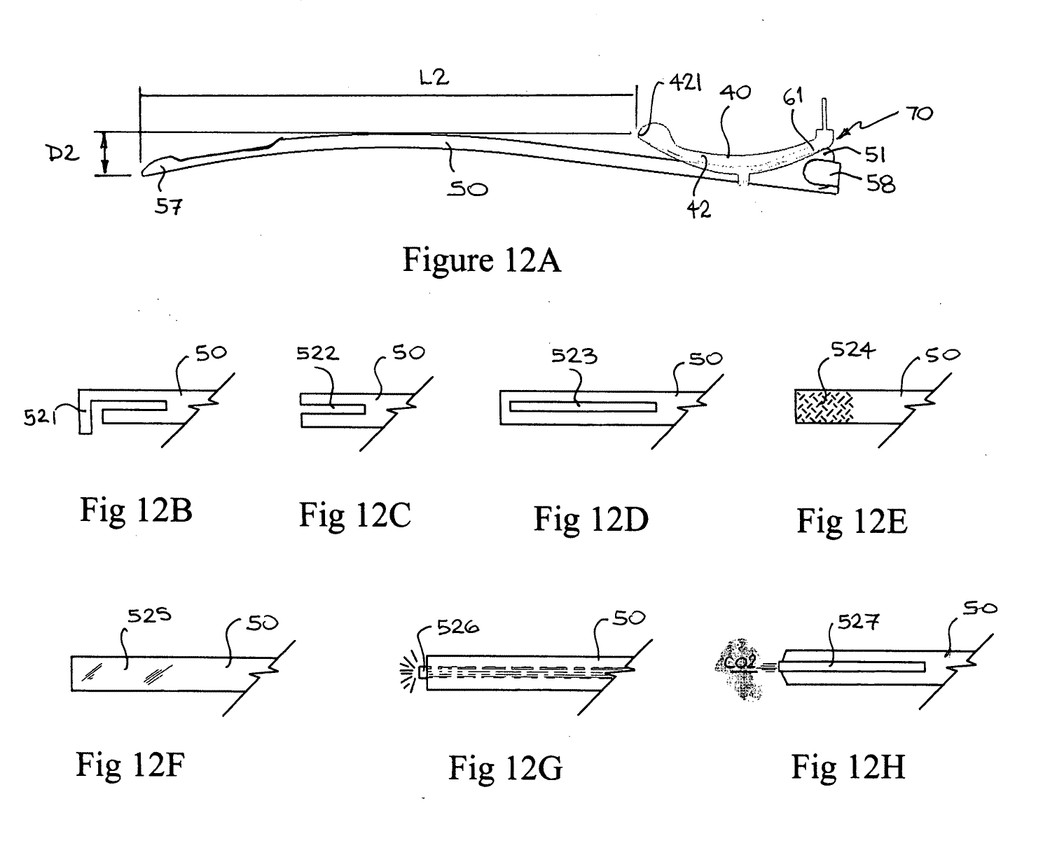

Cusp depressors may be offered in a variety of different lengths and shapes to cater to the specific anatomy of the patient, or to the specific surgical intervention that the surgeon must practice on the target heart valve, or on a part of the cardiac anatomy adjacent to said target heart valve. FIG. 12A illustrates an exemplary variant of the cusp depressor 50 illustrated in FIG. 11B, the latter configured with a bulbous terminal end 57 and a smaller distance D2 below the distal most end 421 of cardiac tissue retractor. As illustrated in FIG. 11C, a cusp depressor may be also alternatively configured with a predetermined and desirable offset distance W relative to the longitudinal axis 29 of tissue retracting apparatus 1. Cusp depressors may be offered in a variety of classified length and shapes so that the surgeon can select from the surgical armamentarium the most suitable cusp depressor geometry for a given surgical procedure and patient's specific anatomy.

Depressor 50 may be fabricated from surgical grade stainless steel, titanium or a plastic material suitable for surgical use. Alternatively, it may be fabricated from a malleable material allowing the surgeon to bend and shape the cusp depressor as needed based on the patient's specific anatomy, or the amount of cusp retraction or depression required. Alternatively still, the cusp depressor may be fabricated from shape memory alloy allowing it to transform its shape once it is coupled to a cardiac tissue retractor 40. Alternatively still, the cusp depressor may be fabricated from a malleable shape memory that will allow the surgeon to bend and shape the cusp depressor in a desired shape, profile, or geometry and then, after use, when the cusp depressor is sent for sterilization prior to repeated use, the cusp depressor will resume its unbent, unshaped original profile due to exposure to the heat from the sterilization cycle.

As illustrated in FIGS. 12B-12H, cusp depressor 50 may be configured with a variety of distal terminal ends 52, in order to facilitate a surgical intervention that a surgeon may practice when said target valve cusp is being depressed or retracted by said cusp depressor. FIG. 12B illustrates a cusp depressor with a hooked terminal end 521 that may be deployed to advantageously hook a cardiac tissue such as a heart valve chord. FIG. 12C illustrates a cusp depressor with a slotted terminal end 522 that may advantageously serve to segregate or sever a cardiac tissue such as a heart valve chord. FIG. 12D illustrates a cusp depressor with an open body terminal end 523 which may enhance flexibility of the cusp depressor for a given cusp depressor width thus making it less traumatic. FIG. 12E illustrates a cusp depressor textured terminal end 524 advantageously serving to enhance friction between said cusp depressor and said valve cusp being retracted or depressed. Alternatively, said texture may include a hydrogel coating or sticky polymeric treatment. FIG. 12F illustrates an optically clear cusp depressor advantageously allowing the surgeon to visually see cusp surface therethrough. FIG. 12G illustrates a cusp depressor configured to include an illuminating or vision-system fiber optic bundle 526 advantageously allowing the heart cavity located beyond the target valve cusp being retracted or depressed to be illuminated for better visualization. FIG. 12H illustrates a cusp depressor configured with a fluid transfer channel therethrough in the nature of a carbon dioxide gas CO2 transfer passageway 527, advantageously allowing CO2 gas to be channeled into the heart cavity beyond the target cusp being depressed with the aim of reducing purging said cavity of oxygen gas during the surgical procedure.

With reference to FIGS. 5B-5D, at least one of the tissue-engaging blades of cardiac tissue retractor 40 (in this case blade 42) is configured with a depressor-to-retractor interface in the nature of a keyway, channel or seat 60. Seat 60 is configured and sized to receive therein depressor 50. In reference to coordinate axis system 59 in FIG. 11A, seat 60 allows a translational movement 54 of cusp depressor distal end 52 generally along a first x-axis, rotational movement 55 about a second z-axis, and rotational movement 56 about a third z-axis while said cusp depressor 50 is movingly engaged with said cardiac tissue retractor 40 at seat 60, but not yet fully seated or locked in position relative to cardiac tissue retractor 40. This ability to move and orient or position distal end 52 of depressor 50 relative to cardiac tissue retractor 40 is advantageous in allowing the surgeon to steer or guide said cusp depressor between adjacent valve cusps of the target heart valve prior to retracting or depressing one of said valve cusps. This ability to locate distal end 52 adjacent a free margin of a valve cusp that is intended to be depressed, and also beyond the plane of the target heart valve, before applying a retraction load to said valve cusp achieves atraumatic cusp retraction or displacement since the surgeon can gently and progressively engage and then retract the valve cusp.

Proximal end 53 of cusp depressor 50 is preferably configured with a manipulating, grasping or handle portion 58 consisting of two opposed flat planar surfaces. Handle portion 58 is appropriately sized so as to be grasped by a common surgical implement such as a forceps, needle drive or like instrument and then manipulated by the surgeon to insert and engage said cusp depressor 50 with said seat 60.

Depressor 50 is configured with a rib or ridge or protrusion or tongue 51 that mates with cooperating depression, slot, or groove 61 in blade 42. Appropriately configured, tongue 51 and groove 61 together cooperate to provide a locking mechanism or means 70 between depressor 50 and blade 42 when the former is placed in its fully seated position relative to said blade 42 (as illustrated in FIGS. 5D, 6, and 12A). Other than tongue 51 in groove 61, a variety of alternative locking means are also possible including frictional tolerance fits, dovetail-type dog in slot, spring-loaded latching mechanism, bayoneted interface, and other like mating geometries effective in retaining said cusp depressor relative to said cardiac tissue retractor during use.

Alternatively, as illustrated in FIG. 11B, locking means 701 may be provided as part of or integral with said depressor-to-retractor interface 60. For instance, an external dimension of cusp depressor 50 may be designed to provide frictional locking with a corresponding internal dimension of the opening 63 in seat 60, when said cusp depressor is sufficiently inserted within opening 63 and achieves its fully seated position.

As illustrated in FIG. 6, with blades 41, 42, and 43 of cardiac tissue retractor 40 deployed to retract cardiac tissue LAWT, and cusp depressor 50 retracting anterior cusp AC of mitral valve MV, the surgeon is able to investigate and diagnose the subvalvular apparatus of mitral valve MV, or observe the inside of the left ventricle LV including the papillary muscles. In the case of a mitral valve repair requiring a replacement or repair of ruptured chordae tendinae, with the cusp depressor deployed the surgeon is able to observe and surgically resect the ruptured chordae, and then measure the length between the papillary muscle and valve cusp free margin in order to size a synthetic chord replacement. In sizing and implanting multiple chordae replacements, for instance, the surgeon needs to have the cusp depressed at certain times (when placing anchoring synthetic chordae to papillary muscle) and have the cusp not retracted at other times (when assessing the coaptation of valve cusp subsequent to a synthetic chord sutured to the cusp). This procedure may be advantageously achieved with a repetitive insertion and withdrawal of cusp depressor from cardiac tissue retractor, without disrupting the independent retraction of cardiac tissue LAWT by cardiac tissue retractor 40. The advantageous cusp retraction according to the present invention allows the surgeon to practice a delicate surgical intervention on either the target valve, or a subvalvular structure thereof, while the cardiac tissue LAWT is independently being retracted by cardiac tissue retractor 40.

Each of said tissue-engaging blades 41, 42, 43 is preferably pivotingly connected to movable linkage mechanism 30 at a separate blade mount location, interface, or joint 413, 423, 433, respectively. As such, said blades may pivot and orient themselves relative to the cardiac tissue being retracted to assume a less traumatic blade orientation. This blade adaptability tends to provide substantially equal or equilibrated reaction loads being applied by each blade to the contacted portion of body tissue being retracted.

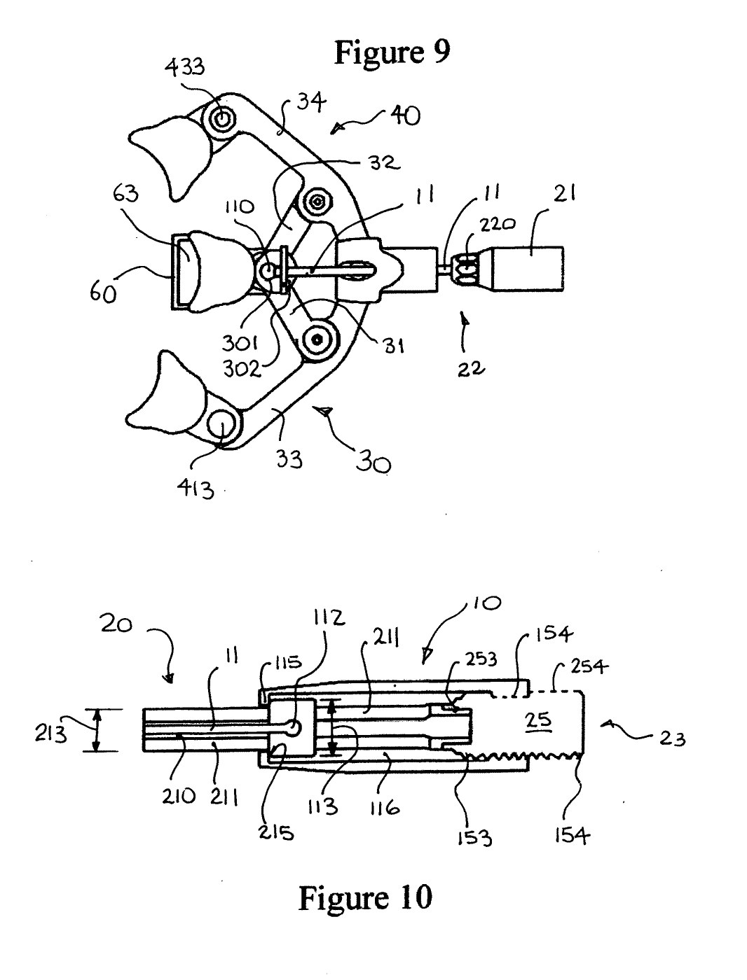

Movable linkage mechanism 30 is comprised of a plurality of movable linkage members. Each linkage member is pivotingly connected or coupled to at least one other linkage member in said linkage mechanism 30. With reference to FIGS. 5C and 9, linkage member 31 is pivotingly connected to linkage member 32 through blade mount joint 423, and pivotingly connected to linkage member 33 at linkage joint 35. Linkage member 32 is pivotingly connected to linkage member 34 at linkage joint 36. Linkage members 33 and 34 are pivotingly connected to each other at linkage joint 37. Generally aligned with blade mount joint 423, linkage mechanism 30 is provided with a socket member 301 configured to receive therewithin ball end 110 of actuating cable 11. As such, linkage mechanism 30 is demountably coupled or connected to actuating cable 11. A locking member, clasp or latch 302 keeps said cable ball end 110 inserted within said socket 301.

Linkage mechanism or assembly 30 is demountable coupled to housing 20 at housing distal end 21 through a housing coupling joint or interface 22 in the nature of a splined mechanical joint 220. Other types of demountable mechanical joints are also possible such as a bayoneted joint, or a threaded joint, or a spring loaded latch joint.

With said linkage mechanism 30 engaged at housing coupling joint 22, a translational movement of cable 11 through housing 20 will entrain a pivoting of the linkage members 31, 32, 33, 34 relative to each other and a simultaneous movement of blades 41, 42, 43 relative to each other. More specifically, retracting cable 11 within said housing 20 will result in mechanical joint 423 being drawn in closer proximity to linkage joint 37 and a spacing apart of blades 41, 42, 43. Conversely, extending cable 11 outwardly for said housing end 21 will result in blades 41, 42, 43 moving closer to each other. As such, linkage assembly 30 is able to articulate in a multitude of different linkage configurations, and consequently able to transmit a multitude of blade spatial geometries or blade spaced apart spatial relationships, relative to said housing 20. As such, tissue retracting apparatus 1 may be adapted or adjusted to take on a desired retraction geometry as blades 41, 42, 43 are selectively moved by actuation cable 11 between a closed blade configuration and an open blade configuration. Linkage mechanism 30 is biased by one or several cooperating spring elements (not shown) acting between adjacent linkage members in a manner to bias the spacing between blades 41, 42, 43 towards a closed blade configuration 44, wherein said blades 41, 42, 43 are in close proximity relative to one another.

Cable 11 is preferably flexible so as to allow flexing of the exposed cable portion extending beyond housing end 21. When blades 41, 42, 43 are engaged with a cardiac tissue to be retracted, a flexible cable provides further adaptability by allowing the entire linkage mechanism 30 to articulate relative to linkage joint 37 and reorient itself as an entire assembly relative to housing 20, in any one given blade configuration (i.e. blade closed, blade open, or intermediately therebetween).

Housing 20 is elongate extending in length along a longitudinal axis 29 between a first housing distal end 21 and a second housing proximal end 23. Housing 20 is substantially hollow and configured with a centrally disposed passageway or channel or bore 210 extending from said distal end 21 towards proximal end 23. Preferably, as illustrated in FIGS. 7 and 8, housing 20 is made from a tubular construction having a cylindrical bore 210, and a cylindrical outer surface over length H1 to facilitate insertion of said housing into stab incision SI formed between two adjacent ribs. Length H1 of housing 20 is sufficiently long to cater for variations in patient anatomy such that when said housing 20 is inserted in said stab incision SI, and said housing 20 is clamped at mounting seat 24 in mechanical joint 962 of instrument positioning arm 96, housing distal end 21 will extend sufficiently beyond the patient's ribcage and into the patient's thoracic cavity TC. A transverse longitudinal slot 211 communicates with said bore 210 over a length H2 of housing 20. Over length H2, housing 20 has a cylindrical external surface interrupted only by slot 211. Slot 211 is configured and sized to slidingly engage with fitting or tongue member 111 of cable 11 when said cable 11 is inserted into said bore 210. Slot 211 also serves as an anti-rotation feature keeping actuating cable 11 from rotating when the latter is translated through said housing 20.

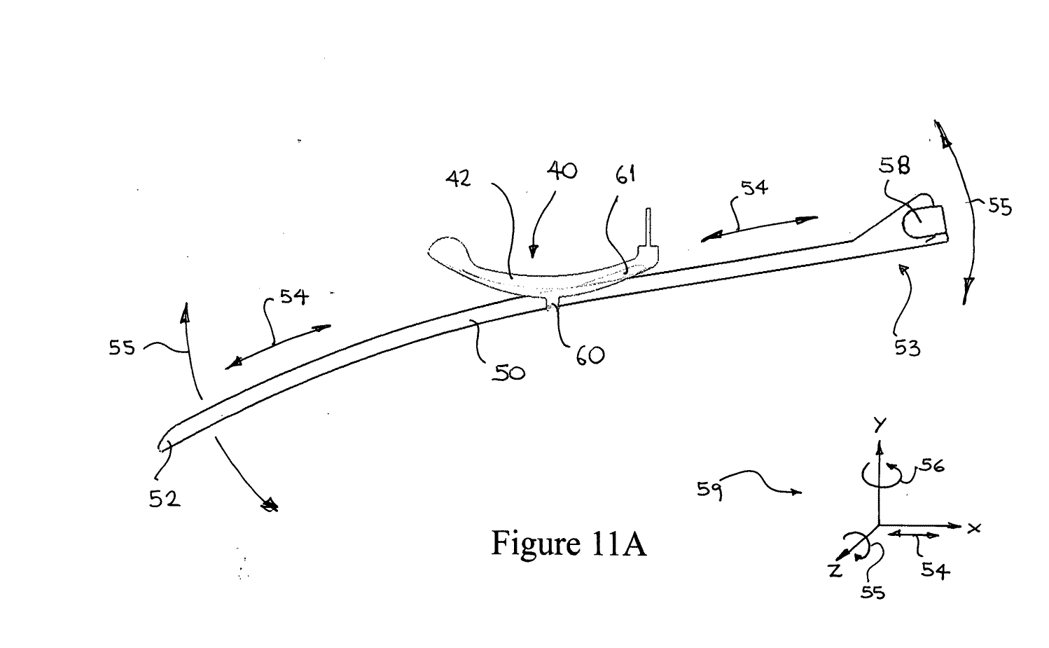

Referring to FIG. 10, at proximal end 23 of housing 20, a threaded member or portion 25 is permanently mounted to said housing, preferably through a permanent joint 253. Joint 253 may be a glued joint, a welded joint, a brazed joint, or any other suitable joint that keeps threaded portion 25 permanently connected to said housing during surgical use. Threaded member 25 is configured with an external thread that mates with internal thread 154 on actuator 10. As such, actuator 10 is rotatingly engaged with housing 20 at said threaded interface 154, 254. When an actuation input is applied to actuator 10, in the nature of a rotational input 100, said actuator 10 is movable relative to said housing between a first threaded position 153 and a second threaded portion 154 (as illustrated in FIG. 4). Said rotational actuation input 100 also results in a movement of actuator 10 along longitudinal axis 29. As well, actuator 10 is slidingly engaged with housing 20 and able to translate or slide relative to said housing over length H2, between a first sliding position 151 (as illustrated in FIG. 5A) and a second sliding position 152.

Length H2 of housing 20 is preferably sized to be between 30 and 70% of housing total length H3, and more preferably to be between 40 and 60% of housing total length H3. As will be described in greater detail below, such housing configuration offers advantages in the deployment of cardiac tissue retractors for valve surgery practiced through an intercostal access port IAP

Actuating member 11 is preferably an elongate flexible cable having a length similar to housing overall length H3. Cable 11 may be of a multi-stranded braided stainless steel construction. At a first distal cable end, cable 11 is configured with an enlarged terminal end, preferably a spherical or ball end 110. Ball end 110 is configured and sized to engage and be demountably coupled to linkage mechanism 30 at socket 301 thereof. As such, actuating cable 11 is coupled to cardiac tissue retractor 40 through linkage mechanism 30 which forms a permanent assembly with said retractor 40. Alternatively, in a variant cardiac tissue retractor 2 comprising a solitary fixed geometry blade 400, and consequently where there is no need for a linkage mechanism, cable 11 may be coupled directly to said blade 400 through a ball-and-socket mechanical interface (not shown) or other like suitable interface. At a second proximal cable end, cable 11 is configured with a key or tongue member 111 in a manner to be preferably demountably coupled to actuator 10. Tongue 111 includes two opposed planar surfaces offset by a predetermined depth to allow tongue 111 to be slidingly engaged in housing slot 211. Tongue 111 may be produced by plastic injection by molding over cable protrusion or enlargement 112 to preferably create a permanent mechanical assembly with cable 11. Alternatively, tongue 111 may be produced by other methods to create an appropriately sized key member to slidingly engage slot 211, or may even be a demountable element of cable 11. The width 113 of tongue 111 is larger than the width dimension 213 of housing 20 over housing length H2 so as to create an tongue abutment face or shoulder 215 that is suitably sized to mate and engage with a cooperating abutment shoulder or surface 115 on actuator 10. Tongue width 113 is smaller than the diameter of actuator internal thread 154 so as to allow cable 11 to be inserted in slot 211 and bore 210 and eventually to allow tongue 111 to be insertable within cavity 116 of actuator 10 at the end of cable assembly process. By having cable tongue 111 fittingly engaged within actuator cavity 116, and by virtue of cooperating abutment shoulders 115, 215, actuating cable 11 can be deployed and translate relative to housing 10 when actuator 10 is actuated over the range of actuator positions. As illustrated and described, cable 11 may be demountable from housing 20, mechanism 30, and actuator 10 in order to allow proper cleaning of bore 210 and allow changeover of cables between surgical uses since such flexible braided cables are difficult to clean and re-sterilize. Alternatively, cable 11 can be permanently mounted to actuator through a mechanical joint allowing relative rotation between actuating cable and actuator when said actuator is deployed between first 153 and second 154 threaded positions.

When actuating member or cable 11 is inserted into housing bore 210 and coupled at first end 110 to linkage mechanism socket 301 and at second end 111 coupled to actuator 10, the following configurations are preferred as a function of actuator 10 position relative to housing 20: when actuator 10 is in first sliding position 151, cable 11 is fully extended from housing 20 and blades 41, 42, 43 are in a blade closed configuration 44; when actuator 10 is in second sliding position 152, linkage mechanism 30 is coupled to housing coupling joint 22 and blades 41, 42, 43 are in a blade closed configuration; when actuator 10 starts to engage a first threaded position 153, blades 41, 42, 43 start to move apart relative to each other away from their blade closed configuration; when actuator 10 engages a second threaded position 154, blades 41, 42, 43 are in a maximum blade open configuration; when actuator 10 engages a threaded position between threaded position 153 and 154, blades 41, 42, 43 take on an intermediate spaced apart blade relationship between their fully closed and fully open blade configuration. An applied actuation input 100 will deploy, adjust, or adapt the plurality of tissue-contacting blades 41, 42, 43 into a desired spatial arrangement suitable for a surgical procedure. Incremental variations in the actuation input 100 will result in a similar incremental variation in said spatial arrangement of said tissue-engaging blades. As such, a surgeon may apply a predetermined actuation input 100 to said actuator 10 to achieve a desired deployment or adjustment of said tissue-engaging blades 41, 42, 43, said spatial relationship of blades 40 being well suited for the retraction of a specific cardiac tissue, a particular surgical incision, or the surgical exposure of an internal cavity.

A housing 20 configuration with features described above is advantageous in surgeries where it is desirable to have an actuation member that is extendible from its housing, for example in valve surgeries practiced through a minimally invasive port access incision, in order to facilitate the coupling of said actuation member with a cardiac tissue retractor. More specifically, with the above advantageous housing configuration, an actuation cable 11 of length similar to housing length H3, said cable end 111 may be extended a considerable length (i.e. a cable extension substantially equal to dimension H2) beyond housing end 21.

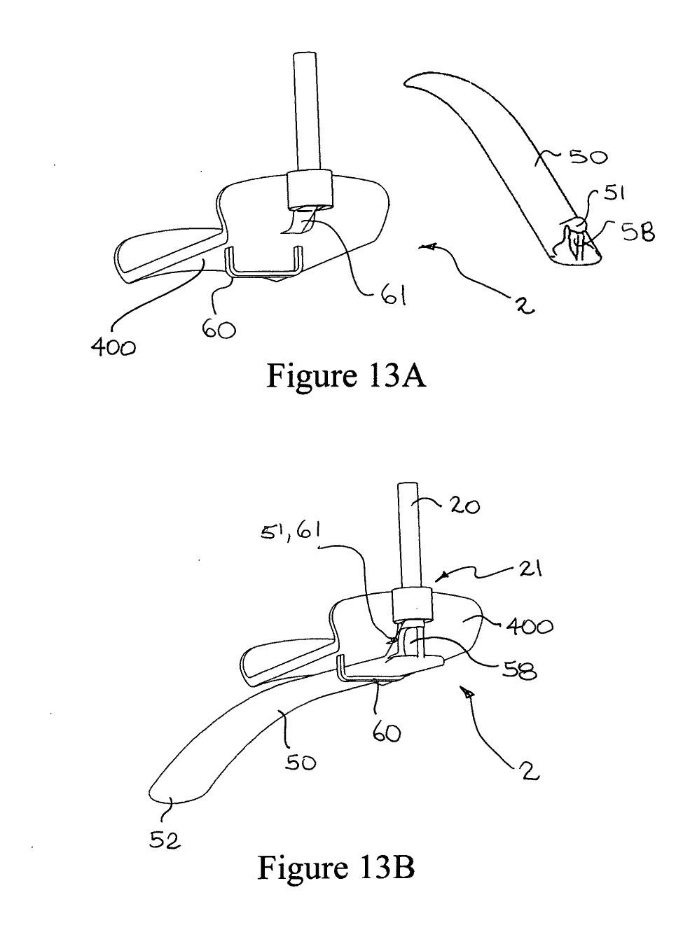

FIG. 13A-13B, according to a second embodiment of the present invention, illustrate a tissue retracting apparatus 2 comprising a single, relatively wider, fixed-geometry cardiac tissue retractor 400 and a couplable cusp depressor 50. The concepts and principles relating to the cusp depressor 50, housing 20, and actuator 10 may be applied to such tissue retracting apparatus as well.

Referring to FIGS. 4 to 6, the deployment of tissue retracting apparatus 1 will be described in greater detail with reference to a surgical method for practicing a surgical intervention on a mitral valve MV, through a left atrial incision LAI and an intercostal surgical approach. The steps include:
performing an intercostal surgical incision between two adjacent ribs of the patient's ribcage to access the patient's thoracic cavity;
inserting blades 971 and 981 of a thoracic retractor 99 into said IAP and deploying said retractor 99 in a manner to engage said blades 971, 981 with patient's ribcage and if as required spreading apart said ribs a desired amount to create an intercostal access port IAP;
exposing the patient's heart HRT as per cardiac surgical procedures practiced through an intercostal surgical approach (i.e. displace lungs, incise pericardium, retract pericardium, mobilize heart within thoracic cavity, etc.);
performing a left atrial or atriotomy incision LAI in the patient's heart HRT, in a manner to obtain a surgical access into the patient's left atrium cavity;
assembling cable 11 into housing 10, and placing actuator 10 in threaded position 154 so that cable ball end 110 extends minimally beyond housing distal end 21;
inserting housing 20 into a separate stab incision SI, located adjacent IAP, in a manner that housing distal end 21 is located within the patient's thoracic cavity;
extending cable end 110 beyond housing end 21 into thoracic cavity TC, and preferably extracorporeally through IAP, by moving actuator 10 to first sliding position 151;
coupling ball end 110 to the assembly consisting of linkage mechanism 30 and cardiac tissue retractor 40 at socket 301;
retracting cable 11 through housing 20 (and drawing into thoracic cavity TC tissue retractor 40) by sliding actuator 10 over housing distance H2 between first sliding position 151 and second sliding position 152;
engaging housing coupling joint 22 between housing 20 and linkage mechanism 30 when actuator 10 begins to rotatingly engage housing threaded portion 25 at a first threaded position 153;
applying a rotational actuation input 100 to actuator 10 to impart a desired spaced apart spatial relationship between blades 41, 42, 43 suitable for deploying cardiac tissue retractor into LAI;
extracorporeally rotating housing 20 about longitudinal axis 29 in a manner that suitably orients the plurality of blades 41, 42, 43 relative to LAI;
proximally and extracorporeally manipulating housing 20 in manner to insert blades 41, 42 and 43 into LAI and place said blades into engagement with left atrium cardiac tissue to be retracted;
adjusting, as necessary, the relative spacing between blades 41, 42, 43 by incrementally and selectively applying an actuation input 100 to actuator 10;
extracorporeally applying a retraction load to housing 20 in a manner to suitably and sufficiently retract the incised left atrial cardiac tissue a desired amount so as to gain surgical access into the left atrium cavity and to the target mitral valve MV;
securing the position and orientation of tissue retracting apparatus 1 (that imparts the above desired retraction load), relative to thoracic retractor 99, by clamping housing 20 at mounting seat 24 to mechanical joint 962 of positioning arm 96;
if required at this point or appropriate, performing a first surgical intervention on the target mitral valve MV such as for instance implanting an mitral annuloplasty ring MAP;
introducing a cusp depressor 50 into IAP and into retracted cavity of left atrium in a manner to engage or couple depressor distal end 52 into seat 60 on blade 42 of cardiac tissue retractor 40;
while engaged with seat 60, orienting and positioning cusp depressor 50 in a manner that distal tip 52 is inserted between adjacent cusps of the target valve MV, or adjacent a free margin of a target cusp to be retracted by said cusp depressor 50;
insert, place or set cusp depressor 50 in its fully-seated position relative to blade 42 and secure its position relative to cardiac tissue retractor 40 through locking means 70 thereby obtaining a desired retraction, depression, or displacement of target valve cusp and surgical access into the left ventricle located beyond the target heart valve MV;
carrying out a surgical intervention on target heart valve MV (for example a cusp resection) or on the subvalvular anatomy or structure of the mitral valve MV located within the left ventricle LV (for example, a chord CRD repair or replacement);
disassembling cusp depressor 50 from cardiac tissue retractor 40 without disrupting the retraction of left atrium cardiac tissue imparted by deployed cardiac tissue retractor 40, and surgically assessing or observing the target valve cusps when the latter are not retracted or depressed by cusp depressor 50;
re-inserting and re-coupling cusp depressor 50 to cardiac tissue retractor 40, as required, to carry out additional surgical interventions on either the mitral valve MV or its subvalvular structure.

The fine tuning of the relative spacing between blades 41, 42, 43 may be carried out at any time during the above process when cardiac tissue retractor is engaged with left atrial cardiac tissue, by incrementally and selectively deploying actuator knob 10 a desired amount.

The invention was described in the context of a cardiac valve surgery performed on the mitral valve MV of the patient. The concepts and principles of the invention may be applied to other tissue retracting apparatus used to perform cardiac surgery on the other cardiac valves (i.e. pulmonary, tricuspid, and aortic), examples of such other retracting apparatus include, but are not limited to:
a cardiac tissue retractor 40 configured and sized to retract ventricular cardiac tissue to gain access into a ventricular heart cavity, and a cusp depressor 50 configured and sized to depress a cusp of an atria-ventricular valve to obtain access to an atrial heart cavity beyond said atria-ventricular valve;
a cardiac tissue retractor 40 configured and sized to retract a right atrium cardiac tissue to obtain access into a right atrium cavity, and a cusp depressor 50 configured and sized to depress a cusp of a tricuspid valve to obtain access to the right ventricle heart cavity beyond said tricuspid valve;
a cardiac tissue retractor 40 configured and sized to retract both a right atrium cardiac tissue and an atrial septum cardiac tissue to obtain access into a left atrium cavity and to the mitral valve through an atrial transeptal approach, and a cusp depressor 50 configured and sized to depress a cusp of the mitral valve to obtain access to the left ventricle heart cavity beyond said mitral valve;
a cardiac tissue retractor 40 configured and sized to retract both a right ventricle cardiac tissue and a ventricular septum cardiac tissue to obtain access into a left ventricle cavity and to the mitral valve through a ventricular transeptal approach, and a cusp depressor 50 configured and sized to depress a cusp of the mitral valve to obtain access to the left atrium heart cavity above said mitral valve;
a cardiac tissue retractor 40 configured and sized to retract a left ventricle cardiac tissue to obtain access into a left ventricle cavity and to the mitral valve, and a cusp depressor 50 configured and sized to depress a cusp of the mitral valve to obtain access to the left atrium heart cavity above said mitral valve;
a cardiac tissue retractor 40 configured and sized to retract a right ventricle cardiac tissue to obtain access into a right ventricle cavity and to the tricuspid valve, and a cusp depressor 50 configured and sized to depress a cusp of the tricuspid valve to obtain access to the right atrium heart cavity above said tricuspid valve;
a cardiac tissue retractor 40 configured and sized to retract a left ventricle cardiac tissue to obtain access into a left ventricle cavity and to the aortic valve, and a cusp depressor 50 configured and sized to depress a cusp of the aortic valve to obtain access to the supravalvular aortic root cavity above said aortic valve;
a cardiac tissue retractor 40 configured and sized to retract a right ventricle cardiac tissue to obtain access into a right ventricle cavity and to the pulmonary valve, and a cusp depressor 50 configured and sized to depress a cusp of the pulmonary valve to obtain access to the pulmonary trunk beyond said pulmonary valve.

1. A tissue retracting apparatus for performing a surgical procedure on a patient's heart, said heart contained within a patient's thorax and beyond a patient's ribcage, said heart being comprised of cardiac tissue, said heart including a plurality of internal heart cavities, each of said heart cavities being delimited in size by said cardiac tissue and also by a target heart valve that controls the passage of blood flow through said heart cavity, said target heart valve including at least one valve cusp movable between a valve-closed and a valve-open configuration to selectively restrict or allow passage of blood therethrough, said tissue retracting apparatus comprising:
a cardiac tissue retractor, said cardiac tissue retractor being configured and sized to retract a portion of said cardiac tissue in a manner so as to provide a surgical access into one of said heart cavities generally through a surgical incision in said cardiac tissue,
a cusp depressor, said cusp depressor being configured and sized to retract said at least one valve cusp of said target heart valve in a manner to allow surgical access beyond said target heart valve, said cusp depressor being operatively couplable to said cardiac tissue retractor through a depressor-to-retractor interface, said cusp depressor able to be secured in a desired cusp-retracting spatial relationship relative to said cardiac tissue retractor through a locking means, whereby, in use, while said cardiac tissue portion is already being retracted by said cardiac tissue retractor, said at least one valve cusp may be retracted sequentially by said cusp depressor when said cusp depressor is operatively coupled to said cardiac tissue retractor.

2. A tissue retracting apparatus according to claim 1, wherein said tissue retracting apparatus is provided with an apparatus-mounting-interface configured to allow mounting of said tissue retracting apparatus to a substantially stable surgical platform, whereby, in use, said cardiac tissue retractor retracts said cardiac tissue portion when said tissue retracting apparatus is securely mounted to said surgical platform at said apparatus-mounting-interface, and said cusp depressor retracts said at least one valve cusp when said cusp depressor is securely mounted to said cardiac tissue retractor through said locking means.

3. A tissue retracting apparatus according to claim 2, wherein said target heart valve is a mitral valve, said surgical platform is a chest retractor engaged with the patient's ribcage, said cardiac tissue retractor is an atrial tissue retractor suitably configured to retract a portion of the left atrium cardiac tissue of the patient's heart, and said cusp depressor is a mitral cusp depressor suitably configured and sized to retract a mitral valve cusp, whereby, in use, while said left atrium cardiac tissue is being retracted by said atrial tissue retractor mounted to said chest retractor, said mitral cusp deflector depresses said mitral valve cusp thereby providing surgical access beyond said mitral valve to the subvalvular apparatus of said mitral valve located within the left ventricle of the patient's heart.

4. A tissue retracting apparatus according to claim 3, wherein said cardiac tissue retractor is comprised of a plurality of tissue-engaging blades, said tissue-engaging blades configured and sized for retracting said left atrium cardiac tissue, said tissue-engaging blades each connected at a blade mount joint of a movable linkage mechanism, said linkage mechanism coupled to an actuator via an actuating member, said tissue retracting apparatus movable between a closed-blade configuration and an open-blade configuration by the actuation of said actuator, wherein in said closed-blade configuration said tissue-engaging blades are in proximity to each other, and in said open-blade configuration said tissue-engaging blades are in a spaced apart spatial relationship, said spaced apart spatial relationship being variably selectable by the degree of actuation input applied to said actuator, at least one of said tissue-engaging blades being provided with said depressor-to-retractor interface, and whereby, in use, said cusp depressor is operatively couplable to said at least one tissue-engaging blade.

5. A tissue retracting apparatus according to claim 4, wherein said linkage mechanism includes a plurality of linkage members, each of said linkage members being pivotingly coupled to at least one other linkage member in said linkage mechanism.

6. A tissue retracting apparatus according to claim 5, wherein said tissue retracting apparatus further comprising a housing, said linkage mechanism coupled to said housing, said housing configured to house at least partially therewithin said actuating member, said actuating member simultaneously coupled to said actuator and to one of said linkage members, whereby when said actuator is actuated, said actuating member moves relative to said housing and entrains the movement of said linkage mechanism so as to move said tissue-engaging blades between said closed-blade and open-blade configuration.

7. A tissue retracting apparatus according to claim 6, wherein said linkage mechanism is pivotingly connected to said housing through at least one of said linkage members, and wherein said actuator is actuated by applying a rotation to said actuator relative to said housing, said applied rotation resulting in a translation of said actuating member relative to said housing, said actuating member translation resulting in a pivoting of said at least one linkage member pivotingly connected to said housing, said pivoting of said at least one linkage member entraining the movement of interconnected plurality of pivotingly-engaged linkage members and the simultaneous movement of said tissue-engaging blades between said closed-blade and open-blade configuration.

8. A tissue retracting apparatus according to claim 6, wherein said linkage mechanism is demountably coupled to said housing at a housing coupling interface and wherein said actuating member is a flexible cable, whereby, in use, said chest retractor is deployed between two adjacent ribs of said patient's ribcage to provide an intercostal access port to the patient's heart, said housing is configured to be insertable though the ribcage via a separate intercostal stab-type port between two adjacent ribs so as to locate said housing coupling interface within the patient's thorax beyond the ribcage, said flexible cable being extendable beyond said housing coupling interface so as to be operatively couplable to said linkage mechanism extracorporeally as said cable extends from said housing coupling interface from within patient's thorax out through said intercostal access port, said linkage mechanism being introduced into patient's thorax through said intercostal access port as said cable once coupled to said linkage mechanism is being retracted within said housing, said cable retraction ultimately entraining the coupling of said linkage mechanism to said housing at said housing coupling interface, said actuator moving said tissue-engaging blades between said closed and open configuration to select a desired blade configuration for left atrium wall retraction when said linkage mechanism is coupled to said housing at said coupling interface and said cable further translates through said housing by deployment of said actuator, said cusp depressor being operatively couplable to said tissue-engaging blade by an insertion through said intercostal access port.

9. A surgical method for performing a surgical procedure on a patient's heart, said heart contained within a patient's thorax beyond a patient's ribcage, said heart being comprised of cardiac tissue, said heart including a plurality of internal heart cavities, each of said heart cavities being delimited in size by said cardiac tissue and also by a target heart valve that controls the passage of blood flow through said heart cavity, said target heart valve including at least one valve cusp movable between a valve-closed and a valve-open configuration to selectively restrict or allow passage of blood therethrough, said surgical method comprising the steps:
incising a cardiac tissue in a manner to obtain open communication into a heart cavity,
engaging a cardiac tissue retractor with a portion of said incised cardiac tissue and retracting said incised cardiac tissue portion sufficiently in a manner to obtain surgical access to an internal heart cavity through said incision and also to a target heart valve delimiting said heart cavity,
operatively coupling a cusp depressor to said cardiac tissue retractor through a depressor-to-retractor interface, and while said cardiac tissue retractor is retracting said incised cardiac tissue portion, deploying said cusp depressor to depress a valve cusp of said target valve in a manner to allow surgical access beyond said target heart valve,
securing said cusp depressor in a desired cusp-retracting spatial relationship relative to said cardiac tissue retractor through a locking means provided between said cardiac tissue retractor and said cusp depressor.
